(19) United States
(12) Patent Application Publication
(10) Pub. No.: US 2026/0000801 A1
(43) Pub. Date: Jan. 1, 2026

(54) Sterilization Tray With Magnetic Drop Latch Arrangement and/or Other Features

(71) Applicant: Zuno Medical, Inc., Campbell, CA (US)

(72) Inventors: Christopher Feezor, San Jose, CA (US); Emile Therault-Shay, San Jose, CA (US); Peter Williams, San Jose, CA (US); Allal McNichol, San Jose, CA (US); Jordan Domanico, San Jose, CA (US)

(21) Appl. No.: 18/831,641

(22) Filed: Jun. 23, 2025

Related U.S. Application Data
(63) Continuation of application No. PCT/US2023/085551, filed on Dec. 21, 2023.
(60) Provisional application No. 63/477,106, filed on Dec. 23, 2022.
(60) Provisional application No. 63/477,147, filed on Dec. 23, 2022.

(51) Int. Cl. A61L 2/24; A61L 2/07; A61L 2/20

(52) U.S. Cl. CPC A61L 2/24; A61L 2/07; A61L 2/20; A61L 2202/122; A61L 2202/123; A61L 2202/14; A61L 2202/24

(57) ABSTRACT

A system can include a valve cap that is positioned above an opening of a sterilization container, the opening of the sterilization container being sized to receive the valve cap. The system includes a valve frame that is affixed to a lid of the sterilization container, and an electro-permanent magnet (EPM) assembly that is releasably and magnetically coupled to the valve cap, the EPM assembly being configured to orient the valve cap in an open configuration via a magnetic field and orient the valve cap in a closed configuration by neutralizing the magnetic field in response to receiving an electrical current.

### CROSS-REFERENCES TO RELATED APPLICATIONS

The present application is a continuation application of PCT/US2023/085551, filed Dec. 21, 2023, which claims priority to U.S. Provisional Application No. 63/477,106, filed on Dec. 23, 2022, and U.S. Provisional Application No. 63/477,147, filed on Dec. 23, 2022, the disclosures of which are herein incorporated by reference in their entirety for all purposes.

### BACKGROUND OF THE INVENTION

Sterilization of items is used in various industries, including health care, pharmaceutical, and food processing industries. A common and proven method used for sterilization applies pressurized high temperature steam in a pressure chamber or vessel for a prescribed period of time. Pressurized high temperature steam within a pressure chamber can be used for sterilization of laboratory equipment and in the industrial manufacturing sector. In hospital and health care environments, laboratory environments, and in the pharmaceutical and food processing industry, sterilization may be accomplished by contacting the item to be sterilized with high temperature steam within a pressure vessel. Alternatively, the item to be sterilized can be contacted with a low temperature sterilizing medium (e.g., ethylene oxide or equivalent low temperature sterilizing medium) in a pressure vessel. Various types of sterilization pressure vessels and autoclave chambers can be used utilized to sterilize items. In many instances, the sterilizing medium is contacted with the item being sterilized.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Apparatus and related methods are provided for sterilizing items (e.g., surgical instruments, instrument trays, implants, and/or implant trays) within a sterilization chamber and subsequent storage and/or transportation thereof until use. An example apparatus includes a container having an internal volume into which items to be sterilized are placed. The apparatus is configurable into an open configuration in which the internal volume is in fluid communication with the surrounding environment. The apparatus (and the items to be sterilized therein) is placed into a sterilization chamber (e.g., an autoclave). The apparatus is configured to reconfigure into a closed configuration in which the internal volume is isolated from the surrounding environment. The reconfiguration to the closed configuration occurs automatically in response to the presence of a vacuum and/or other certain desirable environmental conditions within the sterilization chamber, such as may occur after the completion of a sterilization phase. The sterilization phase can be any suitable portion of a cycle, for example, where the temperature inside the apparatus is greater than a selected sterilization temperature for at least a predetermined amount of time or where a sterilizing vapor or gas fills the internal volume for a predetermined amount of time. The apparatus can remain in the closed configuration with its contents hermetically sealed within the apparatus until when the sterilized items are used, thereby preventing recontamination of the sterilized items prior to use. The apparatus can be reused for sterilizing additional batches of items, thereby providing an effective and economical means to sterilize items and to store and/or transport the sterilized items within a healthcare facility or between healthcare facilities prior to use.

The apparatus disclosed herein can be used for the sterile processing of instrumentation, implants, or other items for a hospital or other healthcare facility. The apparatus may be used for sterile processing at the healthcare facility or at a remote site and transported to a healthcare facility while maintaining the sterile state of the items within the apparatus.

In the operation of certain embodiments of the apparatus disclosed herein, instrument trays and/or other items to be sterilized are placed into a base portion of the apparatus, and a sterilization lid is attached to the base portion. A user arranges the lid or features or mechanisms associated with the lid in an open configuration such that the volume inside the tray is in fluid communication with the surrounding environment. For example, the user can orient a trap door on the lid so that the trap door permits fluid communication through the lid. As examples, a user may lift up on the valve cap to lock the valve in the open configuration, the user may cause power to be provided to an electro-permanent magnet (EPM) which opens the trap door by generating an electro-magnetic force, etc. The valve may be held open by any suitable latch mechanism or force, including, but not limited to, a mechanical, electro-magnetic, or magnetic features.

When the contents of the apparatus are ready to be sterilized, the apparatus is positioned within a sterilization chamber (e.g., an autoclave) with the lid of the apparatus in the open configuration, and a sterilization cycle is initiated. At the beginning or at a particular point after the start of the sterilization cycle, the apparatus becomes sensitive to—and/or begins monitoring—one or more environmental conditions within the chamber. For example, the apparatus can employ any suitable electronic sensor, transducer, etc. for measuring any pertinent environmental condition including, but not limited to, pressure, temperature, and/or humidity. The apparatus can additionally or alternatively include an electronic timer to measure any pertinent time segment, including, but not limited to, time elapsed since reaching a particular threshold and/or duration of conditions within relevant ranges. Furthermore, the apparatus may respond to a particular condition or time based on the status of some other time threshold and/or environmental condition threshold. As examples, the apparatus may begin monitoring temperature after a certain amount of time has elapsed since initiating the sterilization cycle, and/or may track a pressure level or duration of time only as long as a temperature is above a certain threshold or within a certain range.

Once environmental conditions within the sterilization chamber satisfy certain criteria (e.g., pressure reaching a particular sub-atmospheric level after a sufficient duration of exposure to high temperatures has elapsed to confirm adequate sterilization of items), the apparatus is reconfigured from the open configuration to the closed configuration, thereby sealing the apparatus, e.g., by releasing the trap door and/or moving it from the open configuration to the closed configuration. Sealing the apparatus (e.g., closing the trap door) isolates the sterilized items from the outside world and maintains environmental conditions (e.g., pressure and humidity) that existed within the sterilization chamber and equally within the apparatus at the time of reconfiguration, despite additional changes that may occur within the sterilization chamber and/or the external environment. The apparatus can remain sealed until the sterilized items are accessed for use in an operating room.

Thus, in various aspects, an apparatus is provided for sterilizing surgical implements within a sterilization chamber and storing the sterilized surgical implements prior to use. The apparatus includes a container configured to receive one or more surgical implements and one or more valves affixed to the container. The valves are coupled with the container so as to be reconfigurable between an open configuration and a closed configuration. In the closed configuration, the valve and the container at least partially define an internal volume that is sealed. In the open configuration, the valve cap is displaced from the container to form a fluid passage between the internal volume and a volume within the sterilization chamber that is external to the container. The mechanism is configured for selective reconfiguration of the trap door from the closed configuration to the open configuration. The mechanism is configured to automatically reconfigure the trap door from the open configuration to the closed configuration upon completion of a designated portion of a sterilization cycle for one or more surgical implements disposed within the internal volume.

In many embodiments, the container includes a base portion and a top cover that is attachable to and detachable from the base portion. One or more surgical implements can be placed into the base portion and then the top cover attached. The top cover can have an opening that is blocked by the valve cap when the valve is in the closed configuration. When in the open configuration, the valve cap does not block the opening, thereby placing the internal volume of the container in fluid communication with the surrounding environment.

In many embodiments, the apparatus includes one or more spring elements that generate an interface force between the valve cap and the top cover when the valve is in the closed configuration. Such an interface force can ensure compression of an interface seal disposed between the valve cap and the top cover, thereby serving to increase the effectiveness of the interface seal. In many embodiments, the one or more spring elements generate a force on the valve cap that is reacted by the mechanism when the valve is in the open configuration. In various embodiments, when the valve is closed, a vacuum pressure or other condition may exist within the internal volume of the apparatus and provide an additional force that biases the trap door toward the closed configuration.

In many embodiments, the valve includes an latch mechanism that holds the valve in the open position. The latch device may be mechanical, magnetic, or electromagnetic. The valve is configured to be manually displaced by a user to reconfigure the trap door from the closed configuration to the open configuration. The latch device is configured to maintain the valve cap in the open configuration until after completion of the designated portion of the sterilization cycle and/or until environmental conditions within the internal volume satisfy certain criteria.

In many embodiments, the mechanism includes a temperature sensor, a pressure sensor, a electro-permanent magnet (EPM), and a control unit. The temperature sensor can be configured to generate a temperature sensor output indicative of a temperature of the internal volume. The pressure sensor can be configured to generate a pressure sensor output indicative of the pressure within the internal volume. The EPM can be coupled with the latch device and operable to unlatch the valve cap so as to cause reconfiguration of the trap door from the open configuration to the closed configuration. The control unit can be configured to receive the temperature sensor and/or pressure sensor output and control the solenoid. The control unit can be configured to determine if conditions within the internal volume satisfy certain criteria (e.g., corresponding to the completion of the designated portion of the sterilization cycle) and in response to the criteria being satisfied, activate the EPM to unlatch the valve cap, thereby causing the apparatus to reconfigure into the closed configuration.

Any suitable criteria can be used. For example, the control unit can activate the EPM based on the temperature of the internal volume being equal to or greater than a selected sterilization temperature for a suitable period of time and/or based on pressure in the internal volume reaching a predetermined level or a combination of criteria.

In other embodiments, the sterilization container is a sterilization cabinet which is a larger apparatus capable of holding one or more sterilization trays. A sterilization cabinet comprises one or more valve mechanisms, one or more doors which provide access to the internal volume of the cabinet, one or more shelves upon which one or more sterilization trays containing surgical implements are placed, and a control unit with electronic sensors which monitor the environmental conditions within the cabinet and trigger the reconfiguration of the valves from the open to the closed state.

### BRIEF DESCRIPTION OF THE DRAWINGS

FIG. 1 is a flowchart of a method for sterilizing a surgical instrument according to certain aspects of the present disclosure.

FIG. 2 is a schematic of an electro-permanent magnet-based valve cap apparatus in an open position for use in a sterilization container and according to certain aspects of the present disclosure.

FIG. 3 is a sectional side view of an electro-permanent magnet-based valve cap apparatus in an open position for use in a sterilization container and according to certain aspects of the present disclosure.

FIG. 4 is a schematic of an electro-permanent magnet-based valve cap apparatus in a closed position for use in a sterilization container and according to certain aspects of the present disclosure.

FIG. 5 is a sectional side view of an electro-permanent magnet-based valve cap apparatus in a closed position for use in a sterilization container and according to certain aspects of the present disclosure.

FIG. 6 is a sectional side view of an electro-permanent magnet-based valve cap apparatus having a spring for actuating the valve cap according to certain aspects of the present disclosure.

FIG. 7 is a sectional side view of an electro-permanent magnet-based valve cap apparatus having lateral magnets for centering the valve cap according to certain aspects of the present disclosure.

FIG. 8 is an illustration of a sterilization cabinet having an electro-permanent magnet-based valve cap apparatus according to certain aspects of the present disclosure.

FIG. 9 is an illustration of a hybrid electromechanical switch system that can be used to operate an electro-permanent magnet-based valve cap apparatus according to certain aspects of the present disclosure.

FIG. 10 is a block diagram of a control module that can be used to operate an electro-permanent magnet-based valve cap apparatus according to certain aspects of the present disclosure.

### DETAILED DESCRIPTION OF THE INVENTION

Apparatus and related methods are described for sterilizing items (e.g., surgical instruments, instrument trays, implants, and/or implant trays) within a sterilization chamber and subsequent transportation and/or storage of the sterilized items prior to use. For example, a filter-less, reusable sterilization apparatus is described that in an initial configuration (open configuration) provides a pathway to allow the flux of gases (e.g., air, water vapor, etc.) into and out of the apparatus. The apparatus includes a temperature-sensing component, a pressure-sensing component, a humidity-sensing component, and/or a timer, which work together to initiate a reconfiguration of the apparatus to a closed configuration in which the apparatus is hermetically sealed. In many embodiments, the temperature-sensing component monitors temperature of the gases surrounding and/or within the apparatus until a target temperature is reached (e.g., a selected sterilization temperature for sterilizing items within the apparatus). Once the target temperature is reached, the apparatus becomes sensitive to the environmental pressure and/or humidity. Once the environmental pressure and/or humidity reaches a desirable level (e.g., sub-atmospheric and/or low humidity), the pressure sensor initiates reconfiguration of the apparatus into the closed configuration, thereby disrupting the gas pathway and stopping the flux of gases into or out of the apparatus. The apparatus can be kept in the closed configuration and will maintain the environment established within the apparatus at the time the reconfiguration of the apparatus and disruption of the gas pathway (e.g., a sub-atmospheric pressure and/or low humidity state) through a hermetic seal until the contents of the apparatus are accessed for use. When access to the contents of the apparatus is required, the apparatus may either be restored to its initial (open) configuration, which will allow access to the contents directly through the pathway described above, or the apparatus may be put into a third configuration to provide access (e.g., the apparatus's lid is removed).

High temperature steam can be used to sterilize surgical instruments and other medical devices. Steam sterilizes instruments by transferring the heat carried in the water vapor (gas) to the items within the sterilization chamber. This sterilization method exposes the instruments being sterilized to temperatures that generally exceed 120° C. Temperatures in this range may present a challenge to more delicate instruments that may be not able to endure exposure to high temperatures. Some sterilization methods may involve using a gas, a vapor, or a combination thereof as a sterilant to sterilize medical devices much like water vapor or steam can be used. These gases can sterilize instruments at significantly lower temperatures. Some of the gases employed can include ethylene oxide, which disrupts the DNA of microorganisms, hydrogen peroxide plasma, which attacks the membrane lipids, DNA and other cellular components of microorganisms, and ozone, which destroys microorganisms by oxidizing various cellular components. Much like steam, these gases can be introduced into a sterilization container that holds instruments to be sterilized and allowed to dwell within the container for a defined time before being extracted from the container. A vacuum may be generated within the sterilization chamber and container to facilitate the removal of the sterilant. The sterilization container may be compatible with these and any other gas or vapor-based sterilization method in which the sterilant may be injected into a chamber and utilizes vacuum at the conclusion of the sterilization phase to extract the sterilant from the container.

In accordance with aspects of embodiments, a sterilization container may be loaded with instruments and a cover positioned over a container base and latched in position. Valves or trap doors are opened to allow sterilant to flow unobstructed into and out of the container to sterilize instruments during a sterilization process. Following sterilization, a vacuum may be generated within the sterilization chamber to extract the sterilant from the chamber and sterilization container during an aeration phase of the sterilization cycle. Upon concluding the aeration phase, when the chamber begins to rise back toward atmospheric pressure, a control system managing the operation of the sterilization container will trigger the valves/trap doors to close, sealing the container from the external environment prior to the sterilization chamber being opened while maintaining a sub-atmospheric pressure within the container.

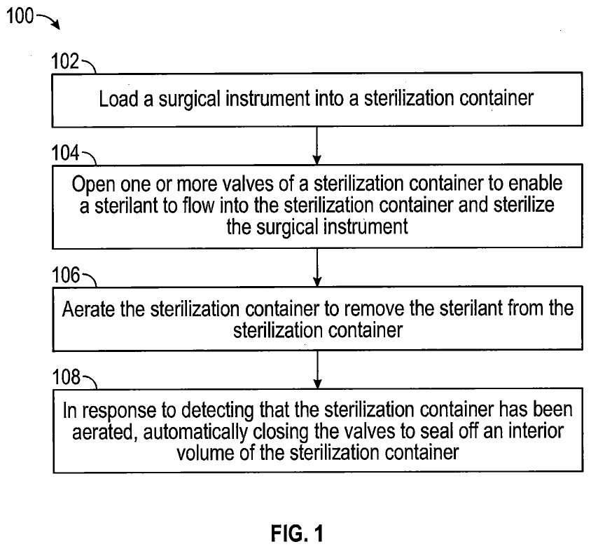

Turning now to the figures, FIG. 1 is a flowchart of an exemplary method for sterilizing a surgical instrument according to certain aspects of the present disclosure.

Block 102 of the method 100 involves loading a surgical instrument into a sterilization container. The sterilization container may include doors that can be opened to insert one or more surgical instruments, and which can be latched shut prior to initiating the sterilization process. In some examples, the sterilization container can include one or more perforated trays that can be used to hold surgical instruments during the sterilization process.

Block 104 of the method 100 involves opening one or more valves of the sterilization container to enable a sterilant to flow into the sterilization container and sterilize the surgical instrument. The sterilant can include a high-temperature steam, a sterilizing vapor, a sterilizing gas, a combination thereof, or any other suitable sterilant. In some examples the one or more valves of the sterilization container may be opened by a control module that may be coupled to the sterilization container.

Block 106 of the method 100 involves aerating the sterilization container to remove the sterilant from the sterilization container. Aerating the sterilization container can involve using generating a pressure gradient within the sterilization container to cause the sterilant to leave the sterilization container. In some examples, aerating the sterilization container may involve generating a vacuum within the sterilization chamber and container via a pump.

Block 108 of the method 100 involves automatically closing the valves to seal off an interior volume of the sterilization container in response to detecting that the sterilization container has been aerated. In some examples, the valves may be automatically closed off by a control module. In some examples, the valves may include electro-permanent magnet-based valve cap apparatus that can be closed by energizing a solenoid to neutralize a magnetic field associated with a permanent magnet in the electro-permanent magnet-based valve cap apparatus.

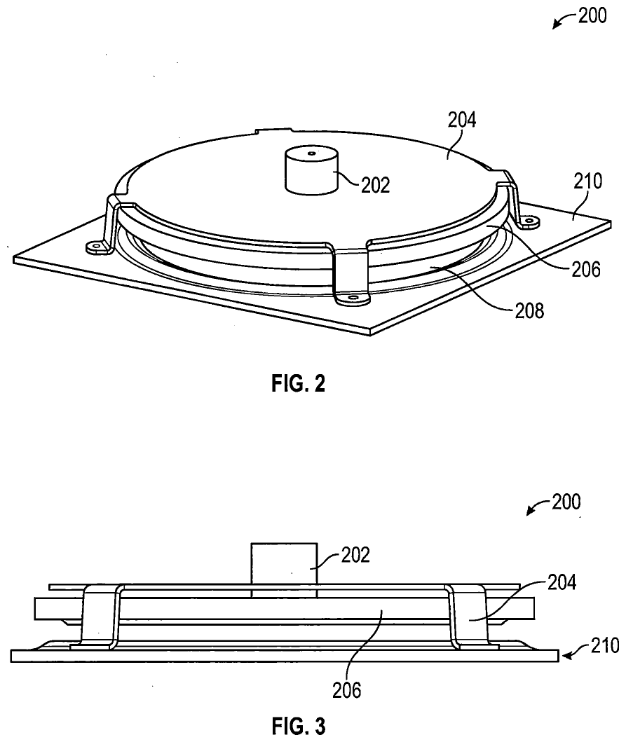

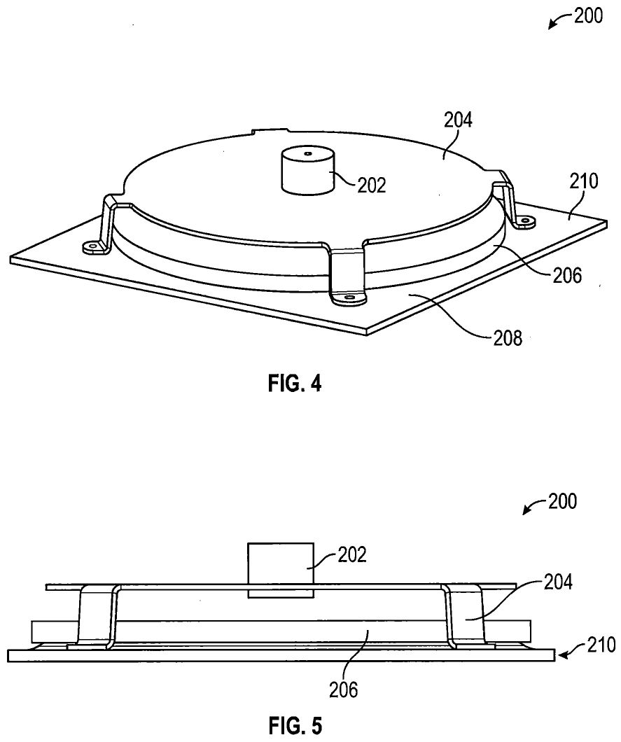

FIGS. 2 and 3 show an example of an electro-permanent magnet (EPM) based valve cap apparatus 200 in an open position for use in a sterilization container 210. Only a top portion of the sterilization chamber is shown in FIGS. 2 and 3, but the structure is known, and example is shown in U.S. Pat. No. 11,826,479, incorporated herein in its entirety. An electro-permanent magnet (EPM) 202 can be affixed to a valve frame 204, which can be mounted to the container 210 over the valve opening 208. For example, the valve frame 204 shown in the figures includes legs that space the valve frame 204 from the container 210, just above the opening 208 in the container. The opening 208 may be anywhere on the container, but in embodiments is on a lid of the container. The valve cap 206 sits between the container 210 lid and the valve frame 204 and can freely float in this space between an open position, as shown in FIGS. 2 and 3, and a closed position, as shown in FIGS. 4 and 5. The examples depicted in FIGS. 4 and 5 may make use of the legs of the valve frame 204 to control lateral motion of the valve cap 206 to ensure that it lands practicably centered on the valve opening 208, thus, creating an effective seal.

An Electro-Permanent Magnet (EPM) is a device that includes a permanent magnet and an electric coil. Under normal conditions, the device acts like a permanent magnet. When a current is passed through the coil, an electromagnetic field can be generated which opposes and, consequently, neutralizes the magnetic force of the permanent magnet. Once the current is removed, the electromagnetic force disappears, and the magnetic force of the permanent magnet can be restored. Thus, the active (i.e. powered) state of an EPM 202 eliminates an existing magnetic field and magnetic force.

The operation of an EPM 202 differs from the operation of a solenoid actuator. A solenoid actuator is an electromechanical device that is used to induce motion of an integrated shaft. When a current passes through a solenoid actuator or it is powered, the device generates a magnetic field which moves the integrated shaft, and anything coupled to the shaft. Once power is removed from a solenoid actuator, the integrated shaft may stop in its current position, or it may return to a default position under the power of a mechanical spring. Thus, a solenoid converts electrical energy into kinetic energy (i.e. mechanical motion) which differs from the EPM 202, which converts electrical energy into magnetic energy to neutralize a permanent magnet. The EPM 202 can operate without any integrated moving components. Thus, any valve assembly that incorporates an EPM 202 can be simpler, more robust, and more reliable structure compared to a solenoid-equipped alternative.

Rather than creating motion of the valve cap 206, the electromechanical valve assembly uses an EPM 202 that can be used to magnetically latch a valve cap 206 in an open position without consuming power. The valve cap 206 may be constructed from a magnetic material or have a component constructed from a magnetic material fixed to the valve cap 206. Prior to initiating a sterilization cycle, the valve cap 206 is latched to the EPM 202 utilizing the passive/non-powered magnetic force of the permanent magnet. At the appropriate time in the sterilization cycle, a current is delivered to the coil of the EPM 202 to neutralize the magnetic force of the permanent magnet allowing the valve cap 206 to be decoupled/released from the EPM 202 and move into another position (e.g. a closed position that may seal the sterilization container 210 on which it is assembled) under the force of gravity or assisted with a spring force.

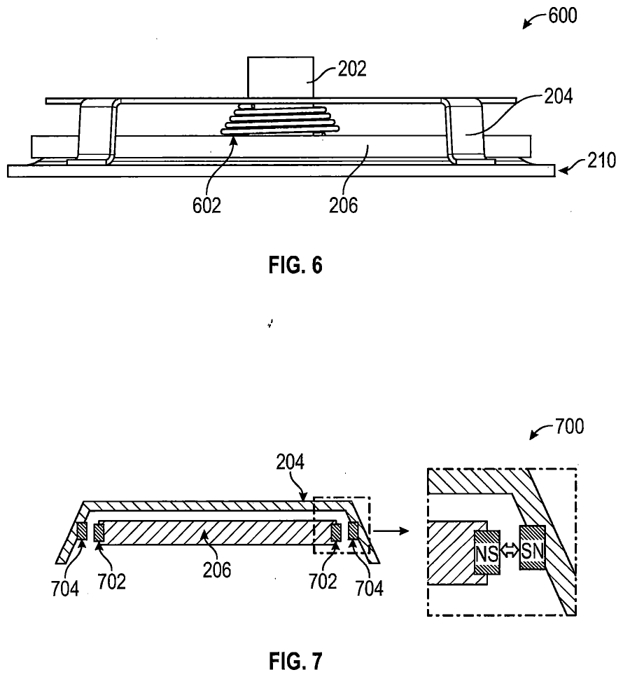

FIG. 6 shows a similar example of the valve structure with the inclusion of a spring 602 that can assist in actuating the valve cap 206 toward a closed position. The spring may exert a spring force that can increase a sealing pressure between the valve cap 206 and a wall of the container 210.

As described above, an EPM 202 functions as a magnet whose magnetic force can be neutralized by generating a magnetic field that opposes and negates the field generated by the permanent magnet. A linear solenoid generates motion of a shaft by generating a magnetic field using an electric coil. The magnetic field is generated by running a current through an electric coil which moves the shaft along the central axis of the coil. The EPM 202 in the current example achieves a result by neutralizing a magnetic field and may not generate motion. A solenoid, as presented previously, generates a magnetic field to induce motion.

In some examples, the EPM 202 unlatches the valve cap 206, thereby allowing it to move due to gravity or through the application of some alternative force. Other examples may include springs as an alternative method for controlling lateral motion of the valve cap 206 and/or ensuring that the valve cap 206 lands in a position that will adequately obstruct the valve opening and seal the sterilization container.

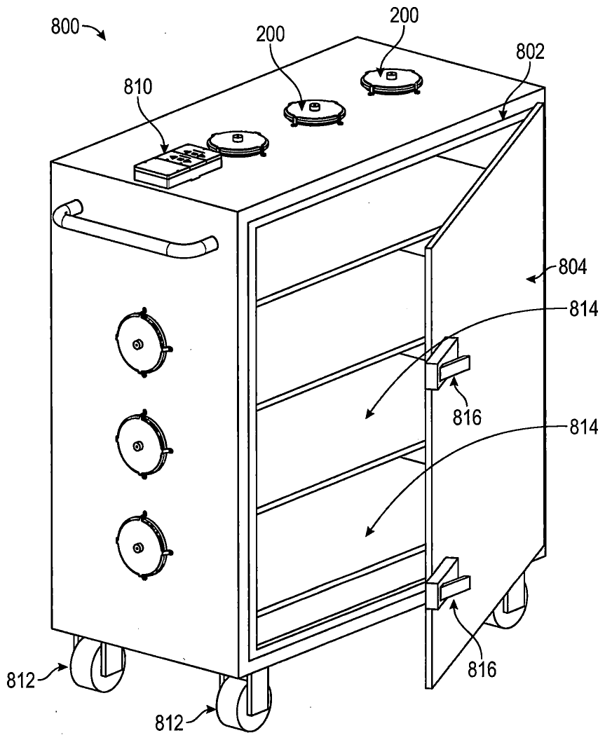

FIG. 7 shows an exemplary valve cap 206 having external lateral magnets 704 that may be mounted on the inner face of the valve frame 204 legs or similar extremities and internal lateral magnets 702 that may be mounted on the edge of the valve cap 206 near each leg of the valve frame 204. The external lateral magnets 704 mounted on the valve frame 204 may have the same polarity as the external lateral magnets 704 mounted on the valve cap 206 so that the internal lateral magnets 702 repel the external lateral magnets 704 and center the valve cap 206 in the valve frame 204 without contact. In some examples, three or more pairs of magnets may be placed around the circumference of the valve cap 206 to satisfactorily control lateral motion of the valve cap 206.

FIG. 8 is an illustration of a sterilization cabinet 800 having an electro-permanent magnet-based valve cap apparatus according to certain aspects of the present disclosure. The example of a sterilization container previously described defines a structure that includes a base portion and a top cover that is attachable to and detachable from the base portion. One or more surgical implements can be placed into the base portion and then the top cover attached. The one or more surgical implements are typically placed in a metal basket or organized in one or more perforated metal trays 814. The basket or tray 814 is then placed within the described container base with the detachable top placed on the base portion to fully enclose its contents. In some examples, the sterilization cabinet 800 may be sized such that only a few (e.g. 1 to 3) baskets or trays 814 may be placed within the sterilization containers.

Some examples of the sterilization container may be larger in size and may contain significantly more basket or trays. The example shown in FIG. 8 of a sterilization cabinet 800 may be best described as a sterilization cabinet 800 in which the door 804 does not detach from the base. Like the top cover of the sterilization container 210, the door(s) 804 provide access to the internal volume of the cabinet 800 for the placement and retrieval of one or more basket or tray(s) which hold the surgical implements. In this example the door 804 replaces the top cover and is located on the side of the container base. The door 804 is hinged on one edge which allows it to swing open to provide access to the internal volume of the cabinet. The door 804 can be outfitted with a gasket 802 around its perimeter to create a seal when the door(s) 804 are closed. The cabinet can be outfitted with one or more latches 816 that hold the doors 804 closed during the sterilization process. The cabinet is also outfitted with one or more electromechanical or electromagnetic valves and a control system as previously described. The valves provide a port which provides an open or unfiltered pathway for the influx and efflux of sterilant between the internal and external volume of the cabinet 800 during the sterilization process. As previously described for the valves affixed to a smaller sterilization container, the electromechanical valve(s) 200 affixed to the wall of the sterilization cabinet 800 close(s) at an appropriate time during the sterilization process, consequently, isolating the internal volume of the cabinet from the external volume and creating a vacuum seal. The valves may be included in any suitable wall of the cabinet, including, but not limited to side walls, top walls, bottom walls, or walls forming at least a portion of a door.

The sterilization cabinet 800 contains the control unit 810 which includes one or more electronic sensors (e.g. temperature sensor, pressure sensor, etc.) that output one or more signals indicative of the environmental conditions within the internal volume of the cabinet 800. In many embodiments, the control unit 810 includes control electronics that monitor the temperature sensor output(s) to identify when the measured temperature(s) are equal to or greater than a selected temperature for sterilizing items within the cabinet 800 and to activate the EPM valve apparatus 200 after a target period of time and/or when particular environmental conditions within the cabinet 800 and/or autoclave exist (e.g., a duration of time required to sterilize cabinet contents, and optionally an additional duration of time to achieve desirable environmental conditions within the cabinet 800). Once the target period of time has passed or particular environment conditions exist, the control unit 810 actuates the EPM valve apparatus 200 to reconfigure the cabinet 800 into the closed configuration, thereby disrupting the gas pathway and stopping the flux of gases into or out of the cabinet 800. The cabinet 800 can then be maintained in the closed configuration until the sterilized items within the cabinet 800 are accessed for use. While the sterilization cycle described is based on the passing of a target period of time, the point at which the EPM valve apparatus 200 is activated can be based on any suitable approach, for example, such as by using the temperature sensor to track the actual temperature profile over time within the interior volume of the cabinet 800 and determining a total sterilization time based on the measured actual temperature profile.

In many embodiments, the control unit 810 includes a pressure sensor that outputs a pressure signal indicative of the internal pressure of the interior volume of the cabinet 800. The control unit 810 can monitor the pressure signal to detect the pressure within the interior volume of the cabinet 800 during the sterilization cycle. After the cabinet 800 is reconfigured to the closed (sealed) configuration within a sterilization chamber (e.g., autoclave), the environmental conditions within the cabinet 800 at the time it is reconfigured into the closed configuration will remain until the seal is broken. For example, a pressure differential between the interior and exterior volume of the apparatus may exist as a result of the interior volume of the apparatus remaining below the pressure of the exterior volume at the conclusion of the sterilization cycle. Without significant entry of air into the cabinet 800, the pressure within the cabinet 800 will remain below the surrounding atmospheric pressure. Therefore, the cabinet 800 can utilize the pressure sensor signal to indicate the loss of seal and can trigger one or more indicators controlled by the control electronics 810 that are used to indicate whether: (1) the interior pressure of the cabinet 800 is below the surrounding atmospheric pressure, thereby indicating retention of the seal; and (2) the interior pressure of the apparatus is not below the surrounding atmospheric pressure, thereby indicating possible loss of the seal. For example, a green indicator light (e.g., a green light emitting diode (LED)) can be lit to indicate that the interior pressure of the cabinet 800 is below the surrounding atmospheric pressure. And the green indicator light can be turned off and/or a red indicator light can be lit to indicate that the interior pressure of the cabinet 800 is not below the surrounding atmospheric pressure. In some embodiments, pressure indicators used to demonstrate integrity of the seal might additionally or alternatively be mechanical.

The functionality described above may be achieved by use of electronics such as microcontrollers or hard logic. A microcontroller can be a small computer on a single integrated circuit containing a processor core, memory, and programmable input/output peripherals. Microcontrollers can be designed for embedded applications, in contrast to the microprocessors used in personal computers or other general-purpose applications. Microcontrollers can be used in automatically controlled products and devices. By reducing the size and cost compared to a design that uses a separate microprocessor, memory, and input/output devices, microcontrollers can make it economical to digitally control many devices and processes. Hard logic can include a combination of electrical components that are operatively connected and designed to perform one or more specific tasks. In contrast, a microcontroller can be programmed to perform different tasks by modifying the programming code and uploading the programming code to the microcontroller. Sterilization apparatuses described herein can use such electronics to perform related functionality described herein including, but not limited to, reading a continuous signal from a temperature sensor, determining when the sensed temperature is equal to or greater than a predetermined target temperature, initiating a timer, and actuating a solenoid after an elapsed period of time to reconfigure a sterilization apparatus into a closed, sealed, configuration.

The sterilization cabinet 800 can be placed inside the sterilizer so that the entire cabinet and its contents may be simultaneously sterilized. Wheels 812 may be fixed to the outside of the sterilization cabinet 800 to enable the sterilization cabinet 800 to be rolled.

Additional examples of the sterilization cabinet 800 utilize two doors that are each hinged on the outer edge and swing in to be latched in the middle of the cabinet. The doors may also slide into the cabinet to provide easy access to the internal volume of the cabinet without the footprint of the door-swing consuming excess space. The door(s) may also swing up with hinges on the top edge of the door or down with hinges on the bottom edge of the door. Additional examples of the sterilization cabinet 800 have doors that may be removed from the cabinet frame or have either removable or hinged doors on multiple faces of the cabinet to provide additional access to the contents inside the cabinet.

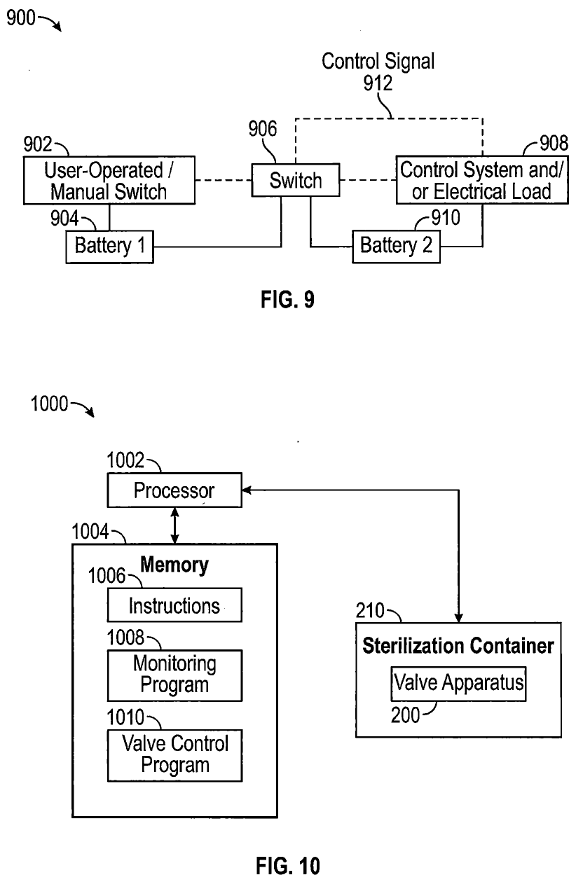

FIG. 9 is an illustration of a hybrid electro-mechanical switch system 900 that can be used to operate an electro-permanent magnet-based valve cap apparatus according to certain aspects of the present disclosure. The hybrid electro-mechanical power switch system 900 may include an interactive switch element 902. In some examples, the interactive switch element 902 can include a button, a capacitive button, a screen, or any other interactive switch element 902. A user can interact with the interactive switch element 902 to create a momentary electrical connection or short to provide a trigger/power/spark to turn on an electronic switch 906 which can power a larger control system or load. Once the user releases the interactive switch element 902, the control system or load remains powered until some alternative event or control signal is given to remove power from the control system.

Initially the system 900 may sit in a steady/sleep state in which no or very little power is being consumed by the entire system. The user-operated or manual switch has been activated which provides power (shown by a solid arrow between the two boxes) and activates/closes the electronic switch. Activation of the electronic switch provides power to the control system or load. The control system or load may then keep the switch activated or closed even though the user-operated or manual switch has been deactivated.

When the control system 908 or load has completed its activities or operations, the control system may send a control signal to the switch 906 deactivating or opening the electronic switch 906 such that that the entire system 900 may return to a steady/sleep state. Implementing the system 900 in a sterilization container that is equipped with electrical and/or electromechanical components for both operation and monitoring purposes may reduce power consumption by creating a process flow that is equivalent to disconnecting the control system from a power source (e.g. the batteries 904 and 910) when the system is not performing one of its prescribed tasks (e.g. checking the status of the container, managing operations during an autoclave cycle, etc.). When a user presses a button, it provides a trigger to power and boot the system, the system then performs the assigned task and powers down. Thus, the system would only consume power while the system is performing a task and power would essentially be disconnected when the system is not performing a task. This should dramatically extend battery life.

Though FIG. 9 depicts multiple batteries 904 and 910, this is not a requirement for operation. A single power source can provide the required power through two different power loops. Any components requiring a constant power supply (e.g. real-time clock or subsystem that is listening for a wireless or other communication signal to wake-up the complete system) can be managed through the use of a standalone battery or utilize an independent power loop through either of the batteries but may not be controlled by either of the depicted switches. Under this scenario, the system consumes power; however, components requiring constant power generally require very little power to perform their required operations.

FIG. 10 is a control module 1000 that can monitor environmental conditions in a sterilization container and can cause valves associated with the sterilization chamber to close according to certain aspects of the present disclosure. The control module 1000 may include a processor 1002 and a memory 1004. The processor 1002 and memory 1004 may be integrated into a single housing or may be distributed from one another.

The processor 1002 can include one processor or multiple processors. Non-limiting examples of the processor 1002 include a Field-Programmable Gate Array (FPGA), an application-specific integrated circuit (ASIC), a microprocessor, etc. The processor 1002 can execute instructions 1006 stored in the memory 1004 to perform one or more operations. In some examples, the instructions 1006 can include processor-specific instructions generated by a compiler or an interpreter from code written in any suitable computer-programming language, such as C, C++, C #, etc.

The memory 1004 can include one memory device or multiple memory devices. The memory 1004 can be volatile or non-volatile, in that the memory 1004 can retain stored information when powered off. Non-limiting examples of the memory 1004 include electrically erasable and programmable read-only memory (EEPROM), flash memory, or any other type of non-volatile memory. At least a portion of the memory device includes a non-transitory computer-readable medium. A computer-readable medium can include electronic, optical, magnetic, or other storage devices capable of providing the processor 1002 with the instructions 1006 or other program code. Non-limiting examples of a non-transitory computer-readable medium include magnetic disks, memory chips, ROM, random-access memory (RAM), an ASIC, a configured processor, optical storage, or any other medium from which a computer processor can read the instructions 1006.

In some examples, the control module 1000 can monitor various environmental conditions that exist within the sterilization chamber and container during a sterilization cycle to track progress through the cycle so that the valves can be closed at the appropriate time. For example, the control module may execute a monitoring program 1008 to monitor the environmental conditions based on received sensor data. In some examples, the output from the sensors may be logged (i.e. stored in non-volatile memory) and queried at a later point to provide performance feedback regarding the sterilization cycle to a user. For example, the data may be queried to determine or report the peak sterilization temperature achieved, the length of time that the sterilization container was exposed to a particular temperature and/or pressure during a sterilization cycle, or other characteristics of the sterilization cycle that may indicate the quality or efficiency of the sterilization cycle.

Modern sterilizers can report these and other parameters from each sterilization cycle that is run, however, the sensors are part of the sterilizer and consequently represent measurements taken inside the sterilization chamber and not inside the sterilization container where the surgical implements reside during sterilization. Consequently, the data represents averages over the entire sterilization chamber. In reality, a gradient of environmental conditions exists within a sterilization chamber (e.g. the local temperature near the drain of a steam autoclave is generally cooler than regions closer to the top of the autoclave) and the exact conditions to which a container is exposed may vary from the values measured and reported by the sterilizer depending on where the container is located within the chamber during the sterilization process. The control module 1000 of a sterilization container collects and records the data from the container on which it is mounted, and this data is specific to that container and the contents of that container.

The control module 1000 may also store diagnostic data related to the number of sterilization cycles that the container has been exposed to, remaining battery life, shelf-life (i.e. time since the container was last sterilized and sealed), or other diagnostic related parameters.

In some examples, the control module 1000 can include a mechanism for uploading data relevant to the specific container associated with the sterilization container and stored as necessary. For example, the information about contents of the sterilization container may be uploaded and stored on the control module 1000. This may provide data that complements or can be integrated with external databases and software that reflect larger scale inventory management and/or EHR (Electronic Health Record)/EMR (Electronic Medical Record) systems that are utilized by hospitals, healthcare facilities, and medical device manufacturers.

The data may be extracted from storage within the control module 1000 and used to provide feedback to a user directly through the user interface (e.g. an LED on the control module 1000 lights up to indicate that appropriate sterilization parameters have been detected) or may be downloaded from the control module 1000 via a wired or wireless connection to the control module 1000 from an external device or computer. Likewise, data uploaded to the control module 1000 from an external device may be uploaded from an external device via a wired or wireless connection.

The control module 1000 can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers, or other network devices may be stored locally and/or remotely, as appropriate. Where a system includes computerized devices, each such device can include hardware elements that may be electrically coupled via a bus, the elements including, for example, at least one central processing unit (“CPU”), at least one input device (e.g., a mouse, keyboard, controller, touch screen, or keypad), and at least one output device (e.g., a display device, printer, or speaker). Such a system may also include one or more storage devices, such as disk drives, optical storage devices, and solid-state storage devices, such as random-access memory (“RAM”) or read-only memory (“ROM”), as well as removable media devices, memory cards, and/or flash cards.

Such devices also can include a computer-readable storage media reader, a communications device (e.g., a modem, a network card (wireless or wired)), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or Web browser. It should be appreciated that alternate embodiments may have numerous variations from that described above. For example, customized hardware might also be used and/or particular elements might be implemented in hardware, software (including portable software, such as applets), or both. Further, connection to other computing devices such as network input/output devices may be employed.

Storage media computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, Electrically Erasable Programmable Read-Only Memory (“EEPROM”), flash memory or other memory technology, Compact Disc Read-Only Memory (“CD-ROM”), digital versatile disk (DVD), or other optical storage, magnetic cassettes, magnetic tape, magnetic disk storage, or other magnetic storage devices, or any other medium which can be used to store the desired information and which can be accessed by a system device. Based on the disclosure and teachings provided herein, a person of ordinary skill in the art will appreciate other ways and/or methods to implement the various embodiments.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions, and changes in the form of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general-purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied—for example, blocks can be re-ordered, combined, and/or broken into sub-blocks. Certain blocks or processes can be performed in parallel.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and all three of A and B and C.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

All references, including publications, patent applications, and patents, cited herein are hereby incorporated by reference to the same extent as if each reference were individually and specifically indicated to be incorporated by reference and were set forth in its entirety herein.

What is claimed is:

1. A system comprising:
a valve cap that is positioned above an opening of a sterilization container, the opening of the sterilization container being sized to receive the valve cap;
a valve frame that is affixed to a lid of the sterilization container; and
an electro-permanent magnet (EPM) assembly that is releasably and magnetically coupled to the valve cap, the EPM assembly being configured to orient the valve cap in an open configuration via a magnetic field and orient the valve cap in a closed configuration by neutralizing the magnetic field in response to receiving an electrical current.

2. The system of claim 1, wherein the system further comprises:
a pressure sensor; and
a control module that is communicatively coupled to the pressure sensor and is configured to transmit the electrical current to the EPM assembly in response to detecting, by the pressure sensor, that a pressure in an internal volume of the sterilization container has reached a threshold pressure.

3. The system of claim 1, wherein the valve cap, while in the open configuration, connects an internal volume of the sterilization container with an external volume such that a sterilant is able to pass through the sterilization container.

4. The system of claim 3, wherein the sterilant is at least one of: high-temperature steam, a sterilizing vapor, or a sterilizing gas.

5-12. (canceled)

13. A hybrid electromechanical switch system for a sterilization container, the system comprising:
an interactive switch element that can be activated by a user;
a control system that is configured to control one or more electromechanical valves of the sterilization container and is coupled to the switch;
a switch that is electrically coupled to the interactive switch element and is configured to transmit a control signal to the switch;
a first battery that is electrically coupled to the switch and the interactive switch element; and
a second battery that is electrically coupled to the switch and the control system.

14. The system of claim 13, wherein the control signal is configured to cause the control system to close the one or more electromechanical valves, thereby sealing the sterilization container from an external environment.

15. The system of claim 13, wherein the control system comprises a control module that is configured to activate an electro-permanent magnet valve apparatus based on one or more determined environmental parameters meeting or exceeding a threshold value.

16. The system of claim 14, wherein the sterilant is at least one of: high-temperature steam, a sterilizing vapor, or a sterilizing gas.

17. A control module for a sterilization container, the control module comprising:
a processor; and
a non-transitory computer-readable memory that includes instructions that are executable by the processor to:
retrieve, from a sensor, one or more measurements associated with one or more environmental conditions within the sterilization container; and
determine, based on the one or more measurements, one or more environmental parameters associated with the sterilization container.

18. The control module of claim 17, wherein the control module is coupled to an external display peripheral that can display parameters associated with a sterilization process.

19. The control module of claim 17, wherein the control module is configured to activate an electro-permanent magnet valve apparatus based on the one or more determined environmental parameters meeting or exceeding a threshold value.

20. The control module of claim 18, wherein a sterilant used in the sterilization process is at least one of: high-temperature steam, a sterilizing vapor, or a sterilizing gas.
